(19) United States
(12) Patent Application Publication
(10) Pub. No.: US 2020/0398053 A1
(43) Pub. Date: Dec. 24, 2020

(54) SYSTEM AND METHOD FOR REGULATING VITAL ENERGY (ZEN QI)

(71) Applicant: ZHEN LI, TORONTO (CA)

(72) Inventor: ZHEN LI, TORONTO (CA)

(21) Appl. No.: 16/940,183

(22) Filed: Jul. 27, 2020

Related U.S. Application Data
(63) Continuation-in-part of application No. 15/703,985, filed on Sep. 14, 2017, now abandoned.

(30) Foreign Application Priority Data
Nov. 12, 2018 (CN) 2019111022859

(51) Int. Cl. A61N 1/36; A61N 1/05

(52) U.S. Cl. CPC A61N 1/36034; A61N 1/36021; A61N 1/0502

(57) ABSTRACT

A system and method are provided for regulating vital energy, or Zen Qi in an organism. The system comprises a positive electrode and a negative electrode coupled to an electronic device or apparatus generating an output current, where the negative and positive electrodes are placed close to or in direct contact with yin and yang body parts, respectively, of an organism. The system further comprises a power supply section, a current control section, data storage and display section, constant current section, over-current protection section, over-voltage protection section and current detection section. By using real-time current detection and over-current and over-voltage protection, the system ensures the safety of organisms during treatment. The system and method regulates vital energy in an organism and provides health benefits including increased saliva, and improved digestion, memory and immune system function, and endocrine system and Musculoskeletal system.

### CROSS-REFERENCE TO RELATED APPLICATION

This application is a continuation-in-part of U.S. application Ser. No. 15/703,985, filed on Sep. 13, 2017, which claims priority to China Application No. 201610907267.8, filed on Oct. 14, 2016. This application also claims priority to Chinese Application No. 201911102285.9 filed on Nov. 12, 2019. All applications are incorporated by reference in their entireties.

### TECHNICAL FIELD

The present invention relates to the field of biotechnology, more specifically to a system and method for regulating vital energy known as Zen Qi.

### BACKGROUND

Zen Qi is the vital energy that comes from parents of an organism when the organism is born and which can be replenished during the life of the organism. According to Taoism, it is believed that vital energy or Zen Qi can be achieved by practice of physical and mental comprehensive cultivation, or Qigong. According to the theory of traditional Chinese medicine, Zen Qi is the most basic material to maintain organism life as all human life depends on Zen Qi.

Ancient and modern Chinese medicine has many discussions about Zen Qi. “The Yellow Emperor's Classic of Internal Medicine” (“the Treatise”) is an ancient treatise on health and disease said to have been written by the famous Chinese emperor Huangdi around 2600 BC. In it, it is stated that, “Previously people led a calm and honest existence, detached from undue desire and ambition, thus vital energy Zen Qi flowed freely; attention is always kept inside, where can illness come from?” Traditional Chinese medicine also teaches that the biological head belongs to the yang, the foot belongs to the yin, back belongs to yang, and abdomen belongs to yin. The head is defined as a positive part and the feet are defined as negative parts according to traditional Chinese medicine. It is also understood that yang and yin body parts have a relative relationship to one another. For example between yin body parts, the feet are considered to be more yin than the abdomen.

The Treatise also points out that Zen Qi comes from the universe and combines with nutrition, then runs within an organism. The Treatise further states that Zen Qi is the vital material running in meridians network and is energy running in meridians network. Thus, meridians network are paths for receiving Zen Qi. It is understood by those skilled in the art of Traditional Chinese medicine that Zen Qi is very important for humans and other organisms.

Zen Qi relates to the movement of electrons in an organism and the meridians network is the path of movement of electrons in an organism. Zen Qi is affected by food we eat, the quality of our breathing through the lungs (including air quality), as well as by the elements that are conducted through the skin from our environment.

If Zen Qi is in proper balance with an organism's needs then the organism will tend to be healthy. If Zen Qi is out of balance with an organism's needs (too little or too much), then the organism will tend to be get sick more easily.

The existing methods of obtaining Zen Qi include taking a holistic approach through Qigong, meditation, cultivation of Buddhism and Taoism, nutrition and exercise. However, for some individuals, such as the disabled, many of these activities are not possible. Thus, a need exists for alternative methods for promoting vital energy or Zen Qi.

Electrotherapy is a general term for describing the application of electrical energy for therapeutic purposes. Such therapies, however, tend to apply electrical waveforms to localized parts of the body or do not take into account the meridians network and other physiological factors for regulating Zen Qi as part of a holistic approach to promoting wellness, preventing disease, or recovering from illness more quickly and effectively. For example, electrotherapy methods have a history of focusing on applying a controllable voltage to the organism, often a pulsed AC voltage with a certain waveform.

In other electrotherapy methods with a focus on pain management, the system used targets the nerves of an organism, as disclosed in U.S. patent Ser. No. 10/384,062, where a current waveform(s) is applied to the nerves with a frequency between 5 Hz and 2 KHz by placing the electrodes on dermatomes close to the pain region.

In another instance, CN110833656 discloses the application of an intermediate frequency system with a constant current output, where the amplitude of the waveform is controlled in order to maintain a constant current output. U.S. Pat. No. 9,037,248 provides a 10 to 2000 Hz frequency current applied using a cuff embedding the anode and cathode, and applied perpendicular or transverse to the nerves, at locations on the body to manage being pain experienced by the subject.

In U.S. Pub. No. 2012/0053648 a system is provided for treating smoking addictions using preferably pulsed stimulation, at low resistance points on the skin (e.g. nose and ear areas) or at acupuncture points on the head. In this disclosure the electrode is configured to be combined with a laser light output and comprises electrode tips in the form of a needle or curved ball with a contact surface of up to about 5 mm to target particular target points in a sequential application of electro stimulation protocols for a total time frame of 10-20 minutes, touching upon multiple acupuncture skin points. Again the application of the electrodes is localized and they are not configured to be suitable for effecting the regulation of Zen Qi in an organism.

There remains a need for a system and method that can regulate the Zen Qi of organism and thereby address overall wellness, as well as a wide range of conditions arising due to imbalances in, or insufficient levels of Zen Qi with a simple to use configuration that does not require significant circuitry modifications, or changing the placement of electrodes in order to have a beneficial effect on a particular condition that needs to be addressed in an organism.

### SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

To achieve the above goal, the present invention provides a system comprising: positive and negative electrodes for connecting to a subject, a power supply section for providing DC power to the system, a current control section, a data storage and display section, a constant current section for providing (delivering) a constant (DC) current to a subject, an over-current protection section and an over-voltage protection section to respectively limit current and voltage levels in real time, and a current detection section to monitor current levels and facilitate the delivery of a desired level of current to the subject. The system is accordingly configured to provide a constant (i.e. non-pulse and continuous), DC current that is less or equal to a safe level of amperage selected by the user to deliver to the subject for as long as needed. The delivery of a constant current selected by the user is thus understood to be a pre-determined constant current, whether it is selected before delivery of said current to the subject, or selected after initiating the delivery of said current to the subject.

The system's configuration is further designed to filter out waveforms by the use of a capacitor positioned in the circuit before the current output point (between the positive electrode and current protection) of the system. In one embodiment, the capacitor is at least 22 uF. By applying a constant (DC) current without waveforms and using the respective over-voltage and over-current protections, the system helps ensure the flow of current through the body of a subject results in a consistent, uni-directional and non-erratic (smooth) ion flow, such that the ions do not reverse their flow. Unlike many systems of the prior art, keeping the ions in channels flowing in the same direction is very important because suddenly changing the direction, or pace of ion flow causes ions collide and react with each other, as well as with the surrounding protein and cells, and can result in damage to the organism.

In one embodiment, the positive and negative electrodes are respectively approaching (close to), or directly contacting the yang and yin parts of the organism (subject), so that the system device or apparatus and the organism form a complete electrical circuit. Multiple pairs of electrodes may be used and therefore applied to different yang and yin parts of the organism's body respectively. Each electrode of a pair of electrodes (understood herein to always comprise conductive material) may have the same or different shapes, so long as any given electrode has a sufficient contact surface for the yang and yin ends of the body it may be connected to.

In another embodiment, the positive electrode is close to or directly contacts one end the of the body of the subject, and the negative electrode is close to, or directly contacts the other end of the body of the subject. In a related embodiment, the positive electrode is close to, or in direct contact with the head of the subject, and the negative electrode is close to, or in direct contact with at least one foot or both feet of the subject (e.g. human, mammal, or other animal) so that the subject and the device (system) form a complete electrical circuit. When the electrode is close to, or directly contacts the head of the subject, it may be on the top of the head, back of the head or on a side of the head.

In yet another embodiment, the positive electrode is close to, or directly contacts the back of the subject, and the negative electrode is close to, or directly contacts the front (abdomen) of the subject. In still another embodiment, the positive electrode is close to, or directly contacts the back of the subject, and the negative electrode is close to, or directly contacts the bottom of the foot of the subject. In yet a further embodiment, the positive electrode is close to, or directly contacts the head of the subject, and the negative electrode is close to, or directly contacts the front side (abdomen) of the body of the subject.

When the electrodes are “close to” a human or other organism's body part there will be a minimal separation, such as that provided by a light woven material, or the hair of the organism. Any matter creating minimal separation between the electrode's conductive material and skin of the organism should be non-electrostatic, such as organic fabrics. The selection of such materials allows the current to still flow from the positive electrode into the organism through its skin at a yang body part, then through the body and out from the skin again at a yin body part (or region) to the negative electrode to complete the circuit.

In a related embodiment, the electrode ends are connected to conductive material to form the electrodes, wherein the conductive material of each electrode provides a sufficient contact surface to ensure that a constant current is applied to one or more meridians (e.g. cutaneous meridians associated with principal meridians) of a subject. In another related embodiment, the conductive material has a sufficient contact surface to provide a constant current to a cutaneous region (or locations) of meridians channels of a subject. FIG. 8 provides an illustration of some cutaneous locations associated with meridians channels in a human subject.

In still another embodiment, the positive electrode is close to, or directly contacts one end or cutaneous region of a meridian channel of the body of a subject, the negative electrode is close to, or directly contacts the other end or another cutaneous region of the same meridian channel of the body of the subject.

In one embodiment, the conductive material of an electrode has a sufficient contact surface to cover the bottom (plantar region) of a foot, which in general will be about 20 cm or more in length in an adult human. In a further related embodiment the conductive material of an electrode has a contact surface placed either on the bottom of the foot or that wraps around a foot. In still another embodiment the conductive material has about a 200 mm (or 20 cm) diameter. In yet another embodiment, the contact surface area or size of the conductive material is about 300 cm², or larger. In still a further embodiment, the contact surface area or size of the conductive material is at least in one dimension 20 cm long. In another embodiment the contact surface area or size of the conductive material is between about 300 cm² to about 1200 cm².

In still another embodiment, the power supply section rectifies an alternating current (AC) or battery or photocell to direct current (DC) for powering other parts of the device.

In a further embodiment, the constant current section produces a constant current of about 5 mA or less for output.

In another embodiment, the current control section is realized by a microcomputer to control current, which receives a command from a user, sets a target current, detects the current, and provides parameters for the constant current section.

In yet another embodiment, the data storage and display section is for storing real-time time or data for various treatment plans of different users.

In one embodiment, the over-current protection section limits the current flowing through each of the organisms to no more than about 5 mA.

In still another embodiment, the over-voltage protection section is used to limit the voltage to no more than about 499.99 volts for the human body, and for animals, the voltage could be limited depending on the height of the animal. Examples of animals that could benefit from the application of the system and method of the present disclosure include pets (e.g. dogs, cats, birds), zoo animals and animals in rescue centers and sanctuaries recovering from harmful experiences.

In a further embodiment, the current detection section detects the current flowing through and being output from the living body in real time. This current detection section helps to ensure that the system can adjust to maintain a (safe) pre-determined constant current, or else shift to deliver another (safe) pre-determined current level that the user considers more appropriate to meet the needs of a subject.

The functionalities of the system and method of the present disclosure, as described above can be achieved by separate discrete components, or by a microcomputer coupled with the discrete components to reduce the size of the system and improve system reliability.

### BRIEF DESCRIPTION OF THE DRAWINGS

The foregoing summary, as well as the following detailed description of presently preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

FIG. 1 is a block diagram of the system of the present invention;

FIG. 2 is a schematic of a circuit of a device of the system of the present disclosure;

FIG. 3 is a microcomputer software programming flow chart for controlling current of the present disclosure;

FIG. 4 is an illustration of one embodiment of the implementation of electrodes of the present disclosure;

FIG. 5 is an illustration of a second embodiment of the implementation of electrodes of the present disclosure;

FIG. 6 is an illustration of a third embodiment of the implementation of electrodes of the present disclosure; and

FIG. 7 is an illustration of a fourth embodiment of the implementation of electrodes of the present disclosure.

FIG. 8 is an illustration of part of the meridians network in a human body, as published at: https://www.pinterest.com/pin/389842911484272496/, and shows some cutaneous locations of associated vessels of meridians channels within the meridians network, not these principal meridians, which are more commonly known.

FIG. 9 is a schematic of an alternative circuit of a device of the system of the present disclosure

To facilitate an understanding of the invention, identical reference numerals have been used, when appropriate, to designate the same or similar elements that are common to the figures. Further, unless stated otherwise, the features shown in the figures are not drawn to scale but are shown for illustrative purposes only.

### DETAILED DESCRIPTION

Unless defined otherwise, all technical and scientific terms used herein have the same meaning as commonly understood by one of ordinary skill in the art to which this invention pertains.

The use of the word “a” or “an” when used herein in conjunction with the term “comprising” may mean “one,” but it is also consistent with the meaning of “one or more,” “at least one” and “one or more than one.” Where only one item is intended the term “one” or similar language is used.

As used herein, the terms “comprising,” “having,” “including” and “containing,” and grammatical variations thereof, are inclusive or open-ended and do not exclude additional, unrecited elements and/or method steps. The term “consisting essentially of” when used herein in connection with a composition, device, article, system, use or method, denotes that additional elements and/or method steps may be present, but that these additions do not materially affect the manner in which the recited component, device, apparatus, system, use or method functions. The term “consisting of” when used herein in connection with a component, device, apparatus, system, use or method, excludes the presence of additional elements and/or method steps. A component, device, apparatus, system, use or method described herein as comprising certain elements and/or steps may also, in certain embodiments consist essentially of those elements and/or steps, and in other embodiments consist of those elements and/or steps, whether or not these embodiments are specifically referred to.

As used herein, the term “about” refers to an approximately +/−10% variation from a given value. It is to be understood that such a variation is always included in any given value provided herein, whether or not it is specifically referred to.

The recitation of ranges herein is intended to convey both the ranges and individual values falling within the ranges, to the same place value as the numerals used to denote the range, unless otherwise indicated herein.

The use of any examples or exemplary language, e.g. “such as”, “exemplary embodiment”, “illustrative embodiment” and “for example” is intended to illustrate or denote aspects, embodiments, variations, elements or features relating to the invention and not intended to limit the scope of the invention.

As used herein, the terms “connect” and related derivatives refer to any direct or indirect physical association between elements or features of the system, apparatus and/or devices of the present disclosure. Accordingly, these terms may be understood to denote elements or features that are partly or completely contained within one another, attached, coupled, contacting, integrated, incorporated, disposed on, joined together, etc., even if there are other elements or features intervening between the elements or features described as being connected. In the case of describing the flow of current according to the present disclosure, where there are intervening elements or features between an electrode and a subject, connections or contact between surfaces of the subject may include direct contact with the skin of a subject, or indirect contact referred to herein as being “close to” one another, where the distance between the electrode/conductive material is sufficiently close to allow for the constant (non-pulse and continuous) flow of current without a break in the electrical circuit formed with the device (system) of the present disclosure and the subject.

The terms “organism” of “subject” as used herein refers to a human or non-human animal, which may include mammals and other air breathing animals which are cared for by humans in different contexts. An aquatic organism or subject may also benefit from the application of the system and method of the present disclosure with suitable modifications that one skilled in the art could produce so as to achieve the functionalities and objects of the present disclosure.

The terms “therapy” and “treatment,” as used interchangeably herein, refer to an intervention performed with the intention of promoting good health and general wellness, as well as alleviating the symptoms associated with, preventing the development of, or altering the pathology of a disease, disorder or condition. Thus, the terms therapy and treatment are used in the broadest sense, and in various embodiments include one or more of the prevention (prophylaxis), moderation, reduction, and/or curing of a disease, disorder or condition at various stages. Subjects in need of therapy/treatment thus may include those already having the disease, disorder or condition as well as those prone to, or at risk of developing, the disease, disorder or condition and those in whom the disease, disorder or condition is to be prevented.

It is contemplated that any embodiment of the compositions, devices, articles, methods and uses disclosed herein can be implemented by one skilled in the art, as is, or by making such variations or equivalents without departing from the scope and spirit of the invention.

### System and Method for Regulating Zen Qi

Regulating Zen Qi (e.g. replenishing Zen Qi) in an individual is to be distinguished from known electro stimulation and therapy systems that seek to have a localized and intermittent (non-constant) effect on the body using focused point of contact type electrodes (e.g. needles and ball tips), waveform and pulsed energy inputs. Said prior art systems are also not seeking to create a circuit for the flow of current that includes the subject and device/system of the present disclosure. The circuit created according to the present disclosure is based on the ability of current to flow along the body's meridians network and not simply at certain localized points where a subject may be experiencing symptoms.

According to published information about the meridians network as referenced at (https://en.wikipedia.org/wiki/Meridian_(Chinese_medicine)#12_standard_meridians), the meridians network is typically divided into two categories, the jingmai ( [symbol] ) or meridian channels and the luomai ( [symbol] ) or associated vessels (sometimes called “collaterals”). The jingmai contain the 12 tendinomuscular meridians, the 12 divergent meridians, the 12 principal meridians, the eight extraordinary vessels as well as the Huato channel, a set of bilateral points on the lower back whose discovery is attributed to the ancient physician Huato. Within the 12 principal meridians, there are six yin meridian channels that are located in the inner region of the arms, legs, chest and torso and there are six yang meridian channels that are located on the outer region of the arms, legs, head and torso, all of which are well known, but there are also other aspects of the meridians network. Sometimes people mistakenly believe that the 12 principal meridians represent the entire meridians network. Acupuncture points are mainly located in meridian channels. The collaterals contain 15 major arteries that connect the 12 principal meridians in various ways, in addition to the interaction with their associated internal organs and other related internal structures. The collaterals also incorporate a branching expanse of capillary-like vessels which spread throughout the body, namely in the 12 cutaneous meridian regions (or locations) as well as emanating from each point on the meridians channels.

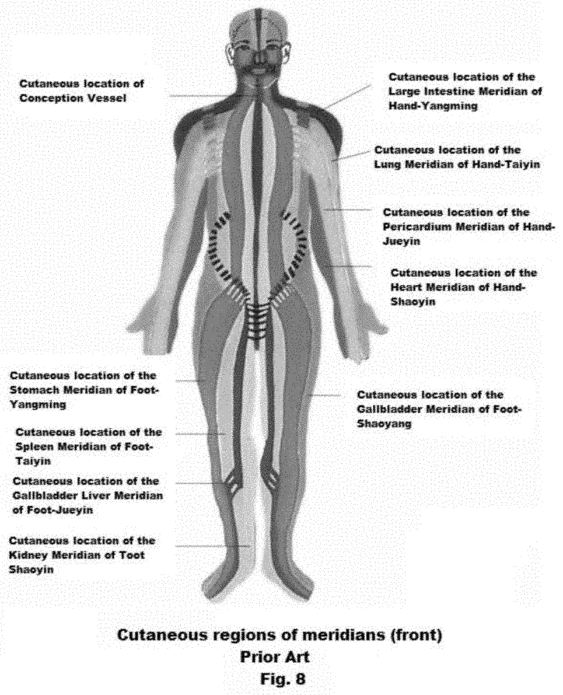

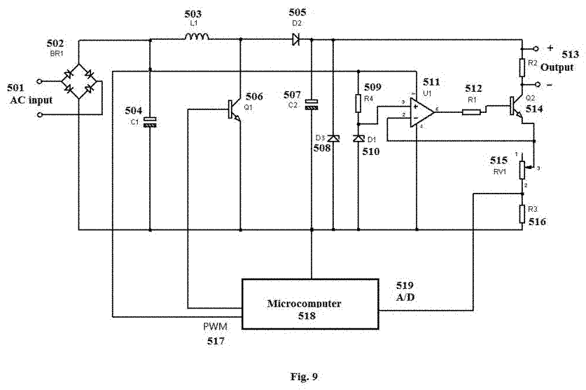

The system and method of the present disclosure mainly targets one or more of the 12 cutaneous regions of associated vessels of the meridians network. Electrons are collected by one of a few of these 12 cutaneous regions at yin body portions of an organism, become anions or stay as electrons, then join the collaterals, go through meridian channels, and are pulled out by the positive electrode from cutaneous regions of the yang body portions of an organism (See FIG. 8 (https://www.pinterest.ca/pin/389842911484272496/). By doing so the system and method of the present disclosure support the achievement of a healthier living body homeostasis. To achieve this cycle of electron movement a certain amount of time, typically longer than what is demonstrated in the prior art systems and methods is needed because the prior art does not consider the make up of the meridians network and how it functions. Accordingly, electrotherapy or electro stimulation of one or more hours is provided for in the present disclosure and can be continued for virtually an unlimited amount of time to meet the needs of an organism.

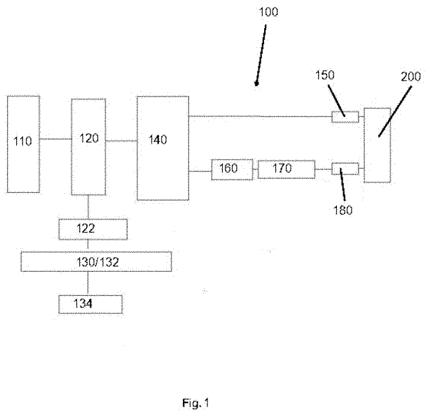

An exemplary implementation of this device and system is described below, as shown in FIGS. 1-9. As stated above, the present disclosure provides a means to promote the flow of vital energy (Zen Qi) through the body of the subject organism by using non-traditional methods. The present disclosure provides a system and method for safely applying a constant electrical current through the body of the subject organism at specific points of contact related to the body's meridians network as understood in accordance with Traditional Chinese Medicine. The safe current through which the human body can flow is 10 mA. In the present disclosure a human subject is generally provided with a constant current of about 5 mA or less, which is a current level less than or equal to half of the safe current. By applying low constant current, the application (delivery) period could extend for at least an hour and carry on for hours at a time on a daily basis to provide continuous stimulation and faster results without harm to the body. For example, some human subjects have received the constant current according to the present disclosure for up to 10 hours a day daily over the course of two or three months.

As shown in FIG. 1, the system 100 one embodiment of the present disclosure comprises a power supply section 110 coupled to a constant current section 120. The constant current section 120 includes current control section 122 which is coupled to a microcomputer 130. The microcomputer 130 includes data storage or memory 132 and is coupled to a display 134 from which a user is capable of inputting data. The constant current section 120 is coupled to an over-voltage protection section 140, which is further connected to a positive electrode 150 on one end; and an over-current protection section 160, current detection section 170 and a negative electrode 180 on another end. The positive and negative electrodes 150, 180 are coupled to an organism 200 at specific parts thereof, which is described in more detail below. The organism 200 could be a human or an animal.

Still referring to FIG. 1, the power supply section 110 includes a power supply that could be from a utility such as a 110V or 220V source, a battery or a photocell. The power supply section 110 includes a rectifier for converting the power supply, including alternating current (AC), to direct current (DC) for powering the system 100. The AC current is regulated by the constant current section 120 to produce a constant current of about 5 mA or less. This is accomplished by the current control section 122, which is operably coupled to the microcomputer 130. In another embodiment, the constant current can be about 10 mA or less. In still another embodiment, the constant current can be adjusted over the course of one or more treatments between about 0.02 mA to about 5 mA. In a related embodiment, the constant current can be adjusted over the course of one or more treatments between about 0.02 mA to about 3 mA.

In still another embodiment, the constant current is applied to an organism for about an hour or more for a single application period. The application period of a given constant current level will depend on the condition and tolerances of the organism (subject). Generally, however, when the constant current applied is about 5 mA or less, it is possible for the organism to receive a constant current on essentially a continuous basis, 24 hours a day, 365 days a year, as needed. Normally the organism will not feel anything if the current is less than or equal to 1.1 mA for an adult man, and less than or equal to 0.7 mA for an adult woman; The organism should lower the current if they feel muscles contract, pain, or discomfort. After using the system and method of the disclosure for a while, and the condition of the meridians network improves and becomes more open to the flow of vital energy, the organism might feel some tingling on the teeth or feet, where contact is made with the electrodes, some movement inside organs, increased saliva generation, hair and skin becoming brighter, or more luminous.

Depending on the configuration of the electrodes with conductive material, the condition and lifestyle of the organism, the application of constant current to an organism according to the present disclosure can be broken down into time periods adapted for an organism's (subject's) schedule and coinciding with when the organism is engaged in little to no movement, or engaged in movement that will not disrupt the operation of the system according to the present disclosure. Examples of time periods when an organism can readily receive constant current according to the present disclosure include when the organism is sleeping, sitting to eat, read or watch television, working, driving or travelling as a passenger, walking or doing light exercise.

In one embodiment, the system and method of the present disclosure can be applied to an organism for one hour/day, for three hours/day, for five hours/day, for seven hours/day or 10 hours/day. In another embodiment, the system and method of the present disclosure can be applied to an organism for one to 10 hours/day over the course of one week, one month, two months or three months.

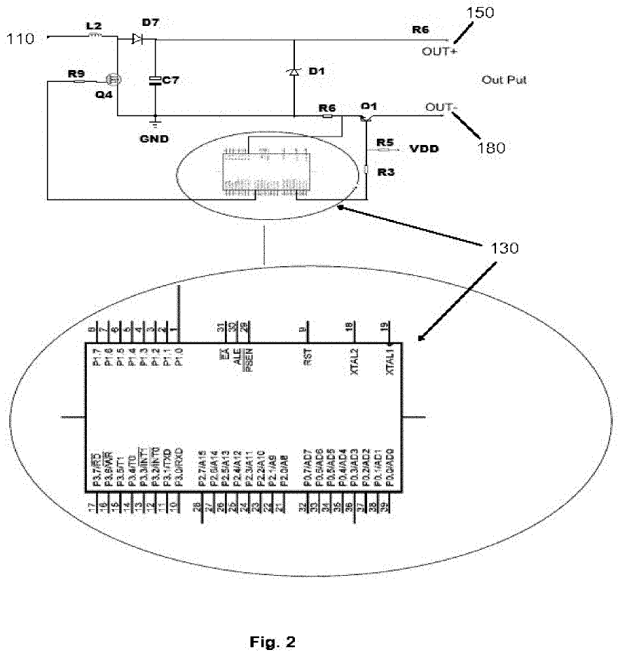

In one embodiment, as shown in FIG. 2, the microcomputer 130 is a 32F429IDISCOVERY type, which is a very comprehensive and powerful computer, with the latest touch-screen technology, sufficient memory 132, and multiple input and output ports. The built-in touch screen 134 can display and key in a user's instructions. The microcomputer 130 also stores and displays real-time data and a variety of treatment plans for different users. Accordingly, the microcomputer 130 is a part of the system 100 that saves the need to add a lot of discrete components. In alternative embodiments, the system can incorporate other single-chip microcomputers such as the STM32 series.

As shown in FIG. 1, the current control section 122 controls the target current by accepting the user's instructions from the display 134 of the microcomputer 130, setting a target current and providing a waveform to the constant current section 120. Furthermore, the current control section 122 provides a signal of on or off to the over-current protection section 160 and the over-voltage protection section 140. In turn, the positive electrode 150 of the output approaches or contacts the head of the human body 200, and the negative electrode 180 approaches or contacts the foot of the human body 200 so that a complete electrical circuit is formed with the system. It is understood herein that reference to the electrodes 150 and 180 also includes the conductive material coupled to, or otherwise integrated with the electrode ends.

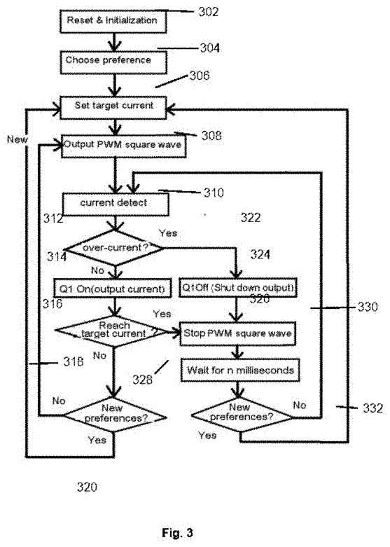

As shown in FIG. 3, a user initiates in one embodiment of the process of the present disclosure by inputting preferences, including a target current, through the display 134 which is coupled to the microcomputer 130.

As shown in FIGS. 2 and 3, the microcomputer 130 generates a controllable waveform for which the current is adjusted by resistor R9 and passed through to transistor Q4, controlling the on and off of transistor Q4. Thus, the charge of capacitor C7 is controlled by inductor L2 while the length of the waveform controls the charging time of capacitor C7 and generates a DC potential in the C7, providing a constant value of the output current for the organism 200. The microcomputer 130 detects real-time current through and output by the organism 200 by way of the resistor R6, such that when the current exceeds a specific target value, the microcomputer 130 controls the gate voltage of transistor Q4. The gate voltage is then set to low, thus the transistor Q4 is turned off, and inductor L2 stops charging capacitor C7. On the other hand, when the current is less than a specific target value, the microcomputer 130 generates a specific length of the square wave pulse-width modulation (PWM) to the gate of transistor Q4, making capacitor C7 charged by inductor L2. This ensures that the current flowing through the organism 200 is near the specific target value at all times.

Referring again to FIGS. 2 and 3, the over-current protection section 160 is used to limit the current flowing through each organism 200 to no more than 5 mA. The over-current protection section 160 comprises resistors R6, R3, R5 and transistor Q1 coupled to the microcomputer 130. The microcomputer 130 detects real-time current flowing through and output by the organism 200 by way of the resistor R6, and when the current reaches a certain value, the microcomputer 130 controls the voltage of transistor Q1, the voltage is set to low, and thus transistor Q1 is turned off. This ensures that a constant target current is outputted.

More particularly with reference to FIG. 2, the transistor Q1 has a base, an emitter and a collector. The collector connects to the negative electrode. A first resistor R6 has a first end connected to the emitter, and a second end connected to the circuit ground (GND). A second resistor R5 has a first end connected to the base of the transistor Q1 and a second end connected to power supply VDD. A third resistor R3 has a first end connected to the base of the transistor and a second end connected to the microcomputer 130. The microcomputer 130 reads the voltage of the emitter of Q1, and then converts this voltage to a current value in real time. The microcomputer 130 compares this current value with the pre-set current value and if the real-time current value is greater than the pre-set current value, then microcomputer 130 outputs a low level of voltage to shut down the transistor Q1, which in turn results in the circuit being shut down so that no current will pass to the organism and in this manner the organism is protected from an over-current.

Referring to FIG. 3, a flow chart for controlling current of an embodiment of the present disclosure is shown. The user resets and initializes 302 the microcontroller and chooses preferences 304, such as setting a target current 306, by inputting the same on the display 134. The microcomputer 130 initiates and generates an output PWM square wave 308 based on the target current. Current is detected 310 by the current detection section 170 for over-current 312. If there is no over-current, transistor Q1 remains on 314 and whether the target current is reached is monitored 316. If the target current is not reached and new preferences are not inputted, the PWM square wave continues to be generated 318 and the current detection loop is continued. In the alternative, a new target current could be inputted 320 and the current detection loop could be restarted with a new PWM square wave output 308. If, however, the target current is reached, the PWM square wave is stopped 326.

Still referring to FIG. 3, if, however, an over-current is detected 322, transistor Q1 turns off 324 and the PWM square wave is stopped 326 for a pre-determined amount of time 328, for example, a few milliseconds. The current detection loop is then restarted 330. If a new target current is set 332, then a new PWM square wave is outputted 308 according to the target current.

Referring to FIGS. 1 and 2, the over-voltage protection section 140 is used to limit the voltage to no more than 499.99 volts. It consists of a Zener diode D1 which is connected in parallel to the output to ensure that the set voltage is not exceeded.

Still referring to FIGS. 1 and 2, the current detection section 170 is integrated with the over-current protection using resistor R6 which provides the microcomputer 130 a voltage that is proportional to current. The microcomputer 130 reads the voltage value and then converts it into a current value. In this embodiment of the present disclosure, resistor R6 has a resistance of 1% accuracy which ensures the accuracy of detection.

Referring to FIG. 9, an alternative circuit of the system of the present disclosure is provided. The AC input 501 and the rectifier bridge capacitor C1 504 constitute the power supply module (section), which rectifies the input AC power into DC for use by other modules (sections). The microcomputer regulates and transistor Q1506 by a PWM (adjustable pulse width) square wave 517. The diode D2 505, the capacitor C2 507, and the inductor L1 503 constitute a voltage conversion module used to convert the low-voltage DC of the two pins of the capacitor C1 504 into a high-voltage DC for other parts of the system. The voltage-regulating diode D3 508 constitutes an over-voltage protection module, such that when the voltage at the terminal exceeds a certain value pre-set by the user, the voltage is stabilized at a certain value to ensure the safety of the organism receiving the constant current.

Operational amplifier U1 511, resistor R4 509, voltage regulator D1 510, resistor R1 512, transistor Q2 514, variable resistor RV1 515, and resistor R3 516 constitute a constant current module. Among them, resistor R4 509 and voltage regulator D1 510 provide a reference voltage for pin 3 of the operational amplifier 511. The characteristics of the amplifier 511 make the voltage values of pins 3 and 2 equal, so that the potential at pin 3 of the variable resistor RV1 515 is always equal to the standard value of the voltage regulator D1 510, such as 3V, thus the current is always equal to the standard value of D1 510 divided by the sum of variable resistor RV1 515 and resistor R3 516. The current flowing through resistor R2 513 is always constant, to provide a constant current output. One can change the resistance of variable resistor RV1 515 to change the magnitude of current, such that the variable resistor RV1 515 constitutes the current intensity setting module. The resistor R2 513 is the load, it presents the object, and its two ends are the output end, the upper side is the positive side (electrode), and the lower side is the negative side (electrode). The pin A/D 519 of the microcomputer is set for signal input, the pin 519, and the resistor R3 516 constitute the current detection module, and the microcomputer 518 converts the detected value between analog and digital to obtain the corresponding current value. The microcomputer 518 constitutes the control module, the overcurrent protection module, the data storage module, the user instruction module, and the data display module. When the current value reaches a certain value, the constant current module feedbacks to the microcomputer 518 through the current detection module, and the microcomputer adjusts the PWM 517. The duty cycle of the square wave or turn off the square wave, so as to play the role of overcurrent protection; the microcomputer can accept the user instruction of settings, including the over current protection value, usage scheme, preference and other data.

To operate the embodiment of FIG. 9, the user turns on the microcomputer 518, the microcomputer resets and initializes, and then the user enters relevant data and sets the value, time, date, name, and preferences of the overcurrent protection. The microcomputer outputs a square wave with a certain duty cycle. For controlling the turning on and off of the transistor Q1 506, a pulse is formed. This pulse charges the capacitor C2 507 through the diode, thereby generating a higher DC voltage for output; the microcomputer 518 detects the current flowing through the resistor R3 516 and if the actual current is large compared to a pre-set current value, the microcomputer 518 will turn off the output of the square wave, otherwise it will turn on the output. The microcomputer will adjust the control of the current according to new instruction values input by a user and will repeat these steps until the user shuts the device/apparatus down.

In other embodiments, the current intensity setting module may also be included in the control module, which is completed by a microcomputer, that is, the microcomputer provides the function of RV1 of the above example. The microcomputer monitors the current of the current detection module in real time to achieve the desired current and regulates the PWM square wave to adjust the duty cycle or turn it off.

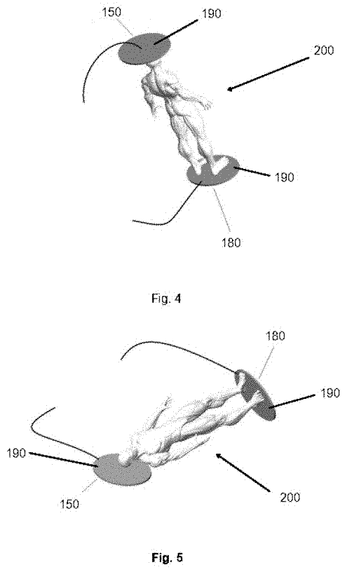

FIGS. 4-6 show examples of the position of the two electrodes 150, 180 on a human subject 200 while the subject is in a standing position (FIG. 4) and a flat lying position (FIGS. 5 and 6). Subjects could receive treatment in other positions as well, while remaining still or while moving provided the operation of the system would not be at risk of being disrupted or damaged.

In these examples, the ends of the positive and negative electrodes 150, 180 are coupled to, incorporate, or are integrated with conductive material 190 that is about 200 mm in diameter or that spans the surface of a side of a human adult's head. The shape of the conductive material, however, could be of other shapes. Also, it is possible to interchange the conductive material (and its supporting structure if applicable) of a given electrode with another conductive material (and its supporting structure if applicable) to accommodate connecting an electrode of the system to another body part, or to allow the organism to receive electrotherapy under different circumstances, for example when awake or sleeping.

In one embodiment of the system, the electrodes/conductive material directly contacts (touches) the skin of a subject. In another embodiment of the system, the electrodes/conductive material indirectly contacts the skin of the subject (e.g. with an intervening material) as a further protection from electric shocks.

The electrode conductive material of the system can be formed in a variety of shapes and with a variety of conductive and semi-conductive materials with different levels of flexibility or rigidity. In an embodiment, one or both of the electrode conductive material may be planar in form. In another embodiment, the conductive material can be covered with a woven material. In a further embodiment, the electrode conductive material can be a circular, triangular, ring, elliptical, square or rectangular shaped. In still another embodiment the electrode conductive material may be irregularly shaped to conform to the dimensions of a structure to support, or conform to the body or part of the body of a subject. In still a further embodiment, the electrode conductive material can be made of copper, or aluminum, or other metals and combinations or alloys thereof. In yet a further embodiment the electrode conductive material may be in the form of a conductive woven fabric, or mesh-like structure that can provide a sufficient contract surface for the flow of current.

In one embodiment, the electrode/conductive material is incorporated into an article of furniture, or furniture accessory (e.g. a table, bed, reclining chair, arm chair, or other seating in a car, train or airplane) to allow the subject to sit down, lay down or stand comfortably. In another embodiment, the electrode is incorporated into an article of clothing (e.g. gloves, socks, shoes, tops, jackets, hats). In still another embodiment, the structure incorporating the electrode/conductive material may be rigid, semi-rigid or flexible enough to form around the shape of a subject's body (or part thereof) to provide a sufficient contact surface. Examples of flexible structures include body forming foams like those used in mattresses or mattress paddings.

The conductive materials 190 of the electrodes make contact (direct or indirect) with the subject 200 with the positive electrode 150 at the head and the negative electrode 180 at the feet (FIGS. 4 and 5) or back area (FIG. 6). The purpose of having a sufficient contact surface for the electrodes 150, 180 (by way of an electrode end being coupled to or integrated with conductive material of a suitable size) is to ensure that sufficient current reaches the yang part of the body to which it is applied and can also run through and out of the yin part of the body flowing along one or more meridians, for example, in a cutaneous region of meridians channels.

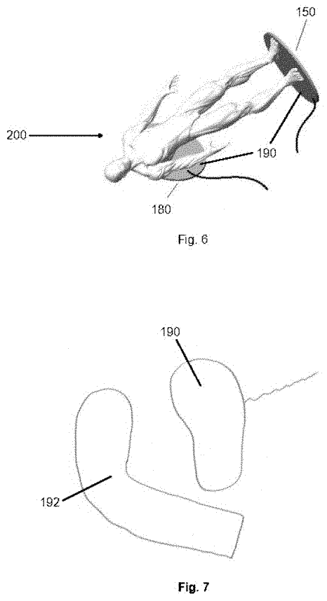

In another embodiment, as shown in FIG. 7, the positive electrode end is coupled to or integrated with a conductive material 190 that is shaped like the bottom of a human's foot. As such, the subject 200 can use it as an insert for a covering such as a sock 192 when being subjected to an embodiment of the method of the present disclosure.

The delivery of electrical stimulation can be by direct contact (touch) with the electrodes 150, 180 (via conductive material 190), or by having the electrodes 150, 180 (with conductive material) just close to the subject 200 without direct contact to the subject's skin. A non-contact application provides for a gentle stimulation. When just close, but not in direct contact with the subject, the electrons come out from the negative electrode 180, transmits to the subject 200 through the air to the skin of the subject, exits the subject 200 through the skin and is transmitted through the air to the positive electrode 150 to complete the circuit. In one embodiment, the negative electrode 180 makes direct contact with the subject's 200 feet or abdomen, and the positive electrode 150 is just close to the subject without direct contact to the subject's skin.

The system 100 of the present disclosure is in one embodiment embodied in a portable device so that the subject could use the same without assistance or supervision. That is, the method of the present disclosure (e.g, in the form of a treatment or therapy) could be self-administered at the subject's home without the assistance of another person (e.g. a therapist, or other operator of the system)

### Uses of the System and Method for Regulating Zen Qi

After being subjected to the method of the present disclosure as described herein, many subjects report that their health condition or sense of well-being has improved including saliva increase, and improvement of digestive and immune system function and memory. The following are actual cases of improved health and well-being due to the application of the system and method of the present disclosure.

Case 1: A 58-year old man suffered with liver cancer and had uncontrollable dull cold pain in the right side of the right upper abdomen for more than 10 years. On the first day of use, with the yin electrode on two feet, setting the current on 3 am for 1 hour, he felt a slight movement and warmness on the right upper abdomen. The positive electrode was made by flexible conductive material wrapping around the liver area, including acupuncture point of Gan shu (Bl-18), with contact surface dimensions of about 60 cm by 20 cm. The negative electrode was made of solid and rigid conductive material and hung on the end of bed so that the feet of the subject could make contact with the conductive material. The subject continued use for five hours a day in a lying down position. After one month of treatment, the subject's liver function improved significantly, abdomen pain reduced significantly, and a CT scan showed that the part of the subject's liver that was swollen had reduced in size from 3 centimeters to 2 centimeters.

Case 2: A 35-year old woman suffered from leukemia and felt pain in her whole body for a long time, including a deep pain in her thigh. The subject received treatment using the system and method described herein for approximately 3 hours a day, setting the current at 0.2 mA, in a lying down position with the positive electrode under the her back and the negative electrode under the plantar of the feet. The positive electrode was made of flexible conductive material and placed under the middle back area of the subject. It was about 60 cm by 20 cm. The negative electrode was integrated into socks and was made of conductive fiber material. After a month of treatment, the pain was relieved and functionality of the white blood cells were significantly enhanced.

Case 3: A 33-year old woman, suffered from fibromyalgia and her whole body was in pain. The first use was for 3 hours in a sitting position with the positive electrode placed near the back area and the negative electrode under the plantar of the feet, with the current set to 0.2 mA. The positive electrode was made of solid (rigid) conductive material connected to the chair she sat in while working. The negative electro was made of solid (rigid) conductive material put under the plantar of the feet on the floor. Each electrode had a contact surface area of about 60 cm by 20 cm. The subject's muscle aches were immediately reduced. Following further use of the system for a week for an hour a day. The subject's muscle aches completely disappeared and the subject was energetic and in a good mood.

Case 4: A male subject suffered from severe muscle atrophy including muscular atrophy of limbs. The subject was severely emaciated and unable to walk. Use was for at least 10 hours a day in a lying position with the positive electrode under the back of the head and the negative electrode under the plantar of the feet and setting the current to 0.1 mA. The positive electrode was made of flexible conductive material placed on top of a pillow. It was about 40 cm by 20 cm in size. The negative electrode was integrated into socks and made of conductive fiber material. After 3 days of use, he felt strengthened, and appetite improved. The subject received therapy continuously following the same regimen for 1 month. The muscles of the hands and feet increased significantly, and the subject gained weight. After continuous therapy for a total of 3 months, the subject was able to walk.

Case 5: An 81-year old woman suffered with Alzheimer's disease resulting in memory loss and lack of ability of self-care. The current was set to 0.1 mA and therapy was conducted lying down with the positive electrode under the back of the head and the negative electrode under the plantar of the feet for 7 hours a day when she was sleeping. The positive electrode was made of flexible conductive material placed on top of a pillow and was about 40 cm by 20 cm. The negative electrode was made of conductive fiber material integrated into socks. After a week, the subject's memory improved and she demonstrated better organization skills. Use of the system continued for a total of three months following the same regimen and she was able to regain memory and take care of herself.

The improvements in health described in each of the cases above are due to the subjects' vital energy having been enhanced. By the using the inventive system and method of the present disclosure, subjects have been able to improve their health in a short period of time as opposed to years of Qigong exercise as traditionally practiced. In fact, many practitioners of standing pile and Qigong, as well as Buddhist and Taoist have used the system and method of the present disclosure and felt that their skills had been improved greatly by using the system and method of the present disclosure as a supplementary tool. Therefore, the present disclosure should be promoted greatly so that all the people can benefit from the same. In sum, the present disclosure supports applications to ameliorate muscle and nerve illnesses and has ameliorative (including therapeutic) effects for symptoms of and the chronic diseases of the brain, as well as wound healing.

It is to be noted that the above are only examples of implementation. According to the principle of the present disclosure, many alternative implementations or improvements could be practiced. For example, other microcomputers, over-voltage protection, over-current protection and current detection could be implemented without departing from the spirit and scope of the present disclosure. As well, the present disclosure could be utilized for humans as well as animals. As such, the described embodiments are to be considered in all respects only as illustrative and not restrictive. The scope of the invention, therefore, will be indicated by claims rather than by the foregoing description. All changes, which come within the meaning and range of equivalency of the claims, are to be embraced within their scope.

1. A system for regulating vital energy in an organism, the system comprising:
a power supply section supplying DC power to the system;
a constant current section configured to supply a pre-determined constant current less than or equal to about 10 mA;
an over-voltage protection section operably coupled to the constant current section, the over-voltage section configured to limit voltage to a pre-determined amount;
an over-current protection section configured to limit current to the pre-determined constant current;
a current detection section operably coupled to the over-current protection section, the current detection section configured to monitor the level of current in the system;
a current control section operably coupled to the constant current section;
a microcomputer operably coupled to the current control section;
a display operably coupled to the microcomputer, wherein a user inputs through the display a target value for the pre-determined constant current; and
a positive electrode for delivering the pre-determined constant current to the organism and with an end connected to a first conductive material with a sufficient contact surface to span at least the size of the top of the head of the organism;
a negative electrode for receiving a current from the body of the organism and with an end connected to a second conductive material with a sufficient contact surface to span at least the size of the bottom of the foot of the organism; and
wherein the negative electrode is positioned to contact a yin body part of the organism and the positive electrode is positioned to contact a yang body part of the organism to form an electrical circuit with the system when the pre-determined constant current is delivered to the organism.
wherein the pre-determined constant current is delivered to the organism for at least an hour.

2. The system of claim 1, wherein the yin body part is the organism's foot or abdomen and the yang body part is the organism's head or back.

3. The system of claim 1, wherein the over-current protection section is configured to limit the pre-determined constant current delivered to the organism to no more than 5 mA.

4. The system of claim 1, wherein the current detection section is configured to detect the current received by the system in real time.

5. The system of claim 1, wherein the microcomputer is configured to generate a controllable waveform to control the opening and closing of a transistor, which controls a charging time of a capacitor by the length of the controllable waveform such that the transistor provides a constant current.

6. The system of claim 1, wherein the over-current protection section comprises:
a transistor first having a base, an emitter and a collector;
a first resistor connected to the emitter;
a second resistor connected to the base;
a third resistor having a first end and a second end, the first end connected to the base; and
wherein the microcomputer having a first pin connected to the emitter, and a second pin connected to the second end of the third resistor.

7. The system of claim 1, wherein the over-voltage protection section comprises a Zener diode connected in parallel to the positive and negative electrodes.

8. The system of claim 1, wherein the sufficient contact surface of the first or second conductive material has an area equal to or greater than about 300 cm².

9. The system of claim 1, wherein the over-voltage protection section is configured to limit voltage to 499.99 volts.

10. A method for regulating vital energy in an organism, the method comprising the steps of:
supplying DC power to a system by a power supply;
supplying a pre-determined constant current less than or equal to about 10 mA by a constant current section;
limiting voltage of the system by an over-voltage protection section;
limiting current supplied by the system to the pre-determined constant current by an over-current protection section;
monitoring current in the system by a current detection section; and
outputting from the system the pre-determined constant current by contacting a positive electrode to a yang body part of the organism and inputting a current to the system from the organism by contacting a negative electrode to a yin body part of the organism to form an electrical circuit with the system.

11. The method of claim 10, further comprising a microcomputer configured to generate a controllable waveform to control the opening and closing of a transistor, which controls a charging time of a capacitor by the length of the controllable waveform such that the transistor provides the non-pulse constant current.

12. The method of claim 10, wherein the over-current protection section comprises:
a transistor first having a base, an emitter and a collector;
a first resistor connected to the emitter;
a second resistor connected to the base;
a third resistor having a first end and a second end, the first end connected to the base; and
wherein the microcomputer having a first pin connected to the emitter, and a second pin connected to the second end of the third resistor.

13. The method of claim 10, wherein the negative electrode has an end connected to a first conductive material with a sufficient contact surface to span at least the area of the bottom of a foot of the organism, and the positive electrode has an end connected to a second conductive material with a sufficient contact surface to span at least the area of the top of the head of the organism.

14. The method of claim 10, wherein the pre-determined constant current is output to deliver said pre-determined constant current to the organism for at least an hour.

15. The method of claim 10, wherein the yin body part is the organism's foot or abdomen and the yang body part is the organism's head or back.

16. The method of claim 10, wherein the pre-determined constant current is less than or equal to about 5 mA.

17. The method of claim 10, wherein the current detection section is configured to monitor the current in the system in real time.

18. The method of claim 10, wherein the over-voltage protection section is configured to limit the voltage to 499.99 volts.

19. The method of claim 13, wherein the sufficient contact surface of the first or second conductive material has an area equal to or greater than about 300 cm².
